(19) United States
(12) Patent Application Publication
(10) Pub. No.: US 2020/0097552 A1
(43) Pub. Date: Mar. 26, 2020

(54) SYNONYM DETERMINATION DEVICE AND SYNONYM DETERMINATION METHOD

(71) Applicant: HITACHI, LTD., Tokyo (JP)

(72) Inventors: Takaaki Haruna, Tokyo (JP); Tadashi Takeuchi, Tokyo (JP); Takuya Oda, Tokyo (JP)

(73) Assignee: HITACHI, LTD., Tokyo (JP)

(21) Appl. No.: 16/524,403

(22) Filed: Jul. 29, 2019

(30) Foreign Application Priority Data
Sep. 26, 2018 (JP) 2018-180949

(51) Int. Cl. G06F 17/27; G06K 9/00; G16H 10/60

(52) U.S. Cl. CPC G06F 17/2795; G16H 10/60; G06K 9/00469; G06F 17/2735

(57) ABSTRACT

Provided is a synonym determination method to improve the determination accuracy of synonyms and reduce data and work. By executing the attribute tag attaching program 21, the CPU 11 attaches an attribute tag to a word extracted from the document data 31. By executing the word co-occurrence index calculation program 22, the CPU 11 calculates a co-occurrence index between words. By executing the attribute-related word search program 23, when there is a set of words having the same attribute among words whose co-occurrence index calculated between words having attributes related with each other is equal to or more than the lower limit value, the CPU 11 determines the set of words to be synonymous. By executing the synonym registration program 24, the CPU 11 presents the words determined to be synonymous to the user 3 as synonym candidates and registers the words presented in the synonym dictionary.

### BACKGROUND OF THE INVENTION

### 1. Field of the Invention

The present invention relates to a synonym determination device and a synonym determination method capable of determining whether words are synonyms.

### 2. Description of the Related Art

In the field of medical treatment or the like, for example, a mistake in an entry of a receipt or an illegal claim is checked with a huge amount of man power while using a computer as a support. In such a check, a keyword search of a document may be performed. In order to improve the search accuracy of the document, it is important for a search keyword to include not only a search term designated by the user but also a synonym for the search term.

U.S. Pat. No. 9,037,464 specification (Patent Literature 1) discloses a learning method for associating a word in a document with numeric representation (appearance probability) in the higher-dimensional space. JP-A-2011-3156 (Patent Literature 2) discloses a method of layer-abstracting and classifying (clustering) data contained in a data set based on values such as similarities, correlation coefficients, and co-occurrence degree between data.

However, in the method disclosed in Patent Literature 1, it is necessary to perform learning using a huge amount of data for associating a word in the document with the appearance probability so as to improve the determination accuracy of synonyms.

In the method disclosed in Patent Literature 2, subordinate concepts having a common superordinate concept are extracted as synonyms. Accordingly, words which are not synonymous are also extracted as synonyms and the amount of human work required to attach sufficient attributes for synonym determination is large so as to improve the determination accuracy of synonyms.

### SUMMARY OF THE INVENTION

In view of the above circumstances, an object of the invention is to provide a synonym determination device and a synonym determination method in which the determination accuracy of synonyms can be improved while reducing the amount of data and the amount of human work.

In order to achieve the above object, a synonym determination device according to the first aspect determines, based on a co-occurrence index between a word having a first attribute and a word having a second attribute related with the first attribute, words having the second attribute in common to be synonymous.

According to the invention, it is possible to improve the determination accuracy of synonyms while reducing the amount of data and the amount of human work.

### BRIEF DESCRIPTION OF THE DRAWINGS

FIG. 1 is a block diagram illustrating a configuration of a document search system according to a first embodiment;

FIG. 2 illustrates a specific example of a word-attribute correspondence table of FIG. 1;

FIG. 3 illustrates a specific example of an attribute relationship table of FIG. 1;

FIG. 4 illustrates a specific example of the result of attaching an attribute tag to a word having an attribute selected from the attribute relationship table of FIG. 1;

FIG. 5 illustrates a specific example of a document-attribute tag correspondence table of FIG. 1;

FIG. 6 illustrates a specific example of a related word correspondence table of FIG. 1;

FIG. 7 illustrates a specific example of a synonym candidate extracted from a word co-occurrence index calculation result of FIG. 1;

FIG. 8 illustrates a specific example of a synonym dictionary of FIG. 1;

FIG. 9 illustrates a specific example of a synonym exclusion list of FIG. 1;

FIG. 10 is a flowchart illustrating a synonym dictionary update processing of the document search system of FIG. 1;

FIG. 11 is a flowchart illustrating an attribute tag attachment processing of the document search system of FIG. 1;

FIG. 12 is a flowchart illustrating an attribute-related word search processing of the document search system of FIG. 1;

FIG. 13 is a flowchart illustrating a synonym registration processing of the document search system of FIG. 1;

FIG. 14 is a flowchart illustrating a document search processing of the document search system of FIG. 1;

FIG. 15 illustrates a specific example of an attribute relationship selection screen displayed on a terminal of FIG. 1;

FIG. 16 illustrates a specific example of a search condition setting screen and a search result screen displayed on the terminal of FIG. 1;

FIG. 17 is a block diagram illustrating a configuration of a document search system according to a second embodiment;

FIG. 18 illustrates a specific example of an original text link table of FIG. 17;

FIG. 19 illustrates a specific example of an original document and a processed document used in the document search system of FIG. 17;

FIG. 20 is a flowchart illustrating a synonym dictionary update processing of the document search system of FIG. 17;

FIG. 21 is a block diagram illustrating a configuration of a document search system according to a third embodiment;

FIG. 22 illustrates a specific example of a logical relationship dictionary of FIG. 21;

FIG. 23 is a flowchart illustrating a synonym dictionary update processing of the document search system of FIG. 21;

FIG. 24 is a flowchart illustrating a co-occurrence index correction processing of the document search system of FIG. 21; and

FIG. 25 illustrates an example of extraction of a synonym candidate of a document search system according to a fourth embodiment.

### DESCRIPTION OF EMBODIMENTS

Embodiments will be described with reference to the drawings. The embodiments described below do not limit the invention according to the claims. In addition, all of the elements described in the embodiments and their combinations are not necessarily essential to the solution of the invention.

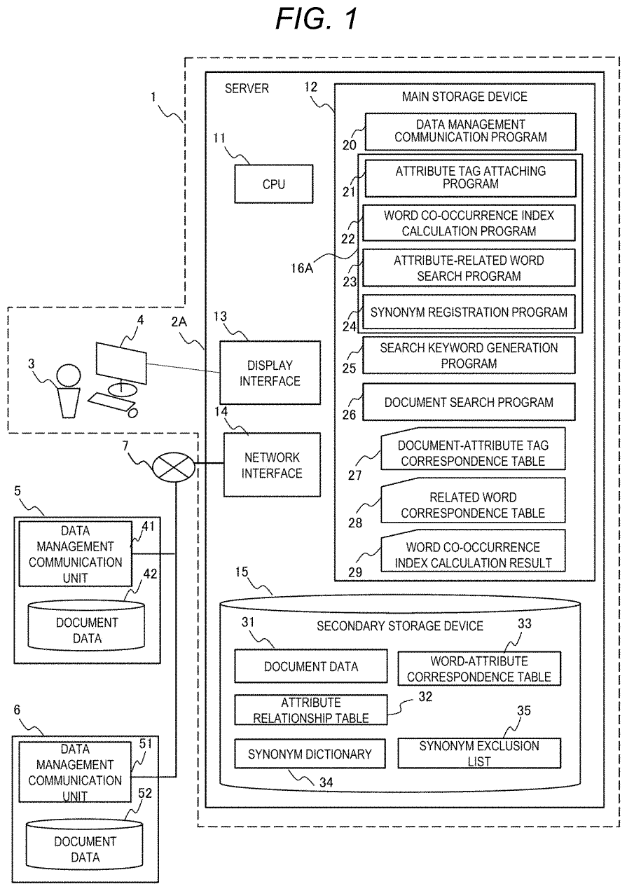

FIG. 1 is a block diagram illustrating a configuration of a document search system according to a first embodiment.

In FIG. 1, a management base 1 includes the document search system. The management base 1 is connected with a remote base 5 and a remote base 6 via a network 7. The network 7 may be a wide area network (WAN) such as the internet, a local area network (LAN) such as Ethernet or Wi-Fi, or a combination of the WAN and the LAN.

The document search system includes a server 2A and a terminal 4. The server 2A determines whether words extracted from a document are synonyms and performs a keyword search of the document. When determining whether the words extracted from the document are synonyms, the server 2A refers to attributes of the words and a co-occurrence index between the words. The co-occurrence index indicates how frequently another word would appear in a sentence when a certain word appears in the sentence. As the co-occurrence index, for example, a word vector distance provided by a word2vec may be used.

When referring to the co-occurrence index between words, the server 2A calculates the co-occurrence index between words having attributes related with each other. Then, when there is a set of words having the same attribute among words having a co-occurrence index equal to or more than a lower limit value, the set of words is determined to be synonymous.

The terminal 4 presents a synonym candidate extracted by the server 2A to a user 3, receives a registration instruction of a synonym from the user 3, receives a search keyword input by the user 3, and displays the search result based on the search keyword.

The server 2A includes a CPU 11, a main storage device 12, a display interface 13, a network interface 14, and a secondary storage device 15. The CPU 11 is hardware that controls the overall operation of the server 2A. The main storage device 12 maybe formed of, for example, a semiconductor memory such as an SRAM or a DRAM. The main storage device 12 may store a program being executed by the CPU 11 and include a work area for the CPU 11 to execute the program.

The display interface 13 is hardware having a function of controlling a display on the terminal 4. The network interface 14 is hardware having a function of controlling communication with the outside. The secondary storage device 15 is a storage device having a large storage capacity and is, for example, a hard disk drive or a solid state drive (SSD). The secondary storage device 15 can hold executable files of various programs and data used for executing the programs.

The main storage 12 holds a synonym dictionary update processing program 16A, a data management communication program 20, a search keyword generation program 25, a document search program 26, a document-attribute tag correspondence table 27, a related word correspondence table 28 and a word co-occurrence index calculation result 29. The synonym dictionary update processing program 16A includes an attribute tag attaching program 21, a word co-occurrence index calculation program 22, an attribute-related word search program 23, and a synonym registration program 24.

The synonym dictionary update processing program 16A determines a set of words to be synonymous based on attributes of words and the co-occurrence index between the words. Then, the words determined to be synonymous are presented to the user 3 as synonym candidates and are registered in a synonym dictionary based on a registration instruction by the user 3.

The attribute tag attaching program 21 attaches an attribute tag indicating an attribute to a corresponding word extracted from document data 31. The word co-occurrence index calculation program 22 calculates a co-occurrence index between words attached with attribute tags indicating attributes related with each other. When there is a set of words having the same attribute among words whose co-occurrence index calculated between words having attributes related with each other is equal to or more than a lower limit value, the attribute-related word search program 23 determines the set of words to be synonymous. The synonym registration program 24 presents the words determined to be synonymous to the user 3 as synonym candidates, and registers the words in the synonym dictionary based on the registration instruction by the user 3.

Information indicating which word of the document data 31 an attribute tag is attached to is registered in the document-attribute tag correspondence table 27. In the related word correspondence table 28, correspondence relationship of words having attributes related with each other is registered. The word co-occurrence index calculation result 29 holds the calculation result of the co-occurrence index between words having attributes related with each other.

The data management communication program 20 performs communication management of data exchanged with the server 2A. The search keyword generation program 25 generates a search keyword used for a document search based on synonyms registered in a synonym dictionary 34. The document search program 26 performs a document search based on the search keyword to which a synonym of a search term input from the user 3 is added.

The secondary storage device 15 holds the document data 31, an attribute relationship table 32, a word-attribute correspondence table 33, the synonym dictionary 34 and a synonym exclusion list 35.

The document data 31 is, for example, text data in which a sentence is described. A data format of the document data 31 may be any format as long as a word search is possible. Relationship between attributes registered in the word-attribute correspondence table 33 is registered in the attribute relationship table 32. Correspondence relationship between a word and an attribute is registered in the word-attribute correspondence table 33. The words determined to be synonyms are registered in the synonym dictionary 34. The words which are in similar scenes in sentences but are not actually synonymous are registered in the synonym exclusion list 35.

The remote base 5 includes a data management communication unit 41 and document data 42. The remote base 6 includes a data management communication unit 51 and document data 52. The server 2A can access the data management communication unit 41 of the remote base 5 and the data management communication unit 51 of the remote base 6 via the network 7. Then, the server 2A can acquire the document data 42 held by the remote base 5 and the document data 52 held by the remote base 6, and store the document data 42 and the document data 52 in the secondary storage device 15.

By executing the synonym dictionary update processing program 16A, the CPU 11 determines, based on the co-occurrence index between a word having a first attribute and a word having a second attribute related with the first attribute, words having the second attribute in common to be synonymous.

For example, by executing the synonym dictionary update processing program 16A, when a first attribute of a first word, and a second attribute of a second word as well as a third word related with the first attribute are given, the CPU 11 determines, based on a first co-occurrence index between the first word and the second word and a second co-occurrence index between the first word and the third word, the second word and the third word to be synonymous.

At this time, the CPU 11 can determine the second attribute related with the first attribute by referring to the attribute relationship table 32. The CPU 11 can determine the first attribute of the first word and the second attribute of the second word as well as the third word by referring to the word-attribute correspondence table 33.

Here, the first word can associate the second word with the third word based on the first co-occurrence index and the second co-occurrence index. Therefore, by determining the second word and the third word to be synonymous based on the first co-occurrence index and the second co-occurrence index, it is possible to improve the determination accuracy of the second word and the third word to be synonymous even when it is not possible to accurately determine the second word and the third word to be synonymous from a third co-occurrence index between the second word and the third word.

Specifically, by executing the attribute tag attaching program 21, the CPU 11 attaches an attribute tag to a corresponding word extracted from the document data 31, and registers the position of the word to which the attribute tag is attached in the document-attribute tag correspondence table 27.

In addition, by executing the word co-occurrence index calculation program 22, the CPU 11 calculates a co-occurrence index between words to which attribute tags indicating attributes related with each other are attached, and stores the result in the word co-occurrence index calculation result 29.

By executing the attribute-related word search program 23, the CPU 11 stores words having attributes related with each other in the related word correspondence table 28 as a set. Then, when there is a set of words having the same attribute among words whose co-occurrence index calculated between words having attributes related with each other is equal to or more than a lower limit value, the CPU 11 determines the set of words to be synonymous.

By executing the synonym registration program 24, the CPU 11 presents the words determined to be synonymous to the user 3 as synonym candidates. Then, when there is a registration instruction from the user 3, the CPU 11 registers the words presented as synonym candidates in the synonym dictionary 34. On the other hand, when there is a non-registration instruction from the user 3, the CPU 11 registers the words presented as synonym candidates in the synonym exclusion list 35.

Here, an attribute of the word-attribute correspondence table 33 can set a general attribute of a word. For example, it is possible to make disease attributes correspond to several thousands to several tens of thousands of disease names, and make medicine attributes correspond to several thousands to several tens of thousands of medicine names. Therefore, it is not necessary to set in detail a sufficient attribute necessary for determining a synonym for several thousands to several tens of thousands of disease names and medicine names, so that the amount of human work for attaching the sufficient attribute necessary for determining a synonym to a word can be reduced. In addition, by using the co-occurrence index between words having the attributes related with each other for determining synonyms, it is possible to reduce the chance that words which are not synonymous are misjudged as being synonymous, and improve the determination accuracy of synonyms.

The execution of the attribute tag attaching program 21, the word co-occurrence index calculation program 22, the attribute-related word search program 23, and the synonym registration program 24 may be shared by a plurality of CPUs or computers. Alternatively, the CPU 11 instructs a cloud computer to execute all or part of the attribute tag attaching program 21, the word co-occurrence index calculation program 22, the attribute-related word search program 23, and the synonym registration program 24 via the network 7, and the execution results may be received.

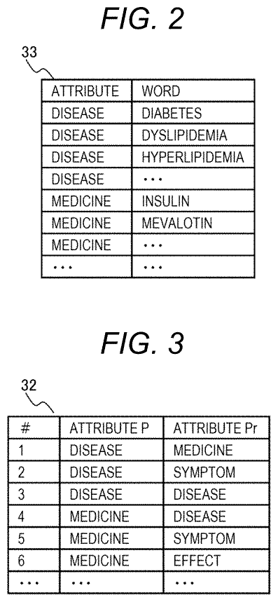

FIG. 2 illustrates a specific example of a word-attribute correspondence table of FIG. 1.

In FIG. 2, correspondence relationship between a word and an attribute is registered in the word-attribute correspondence table 33. For example, a disease attribute is registered corresponding to words indicating a disease name such as diabetes, dyslipidemia, and hyperlipidemia. In addition, for example, a medicine attribute is registered corresponding to words indicating a medicine name such as insulin and mevalotin.

FIG. 3 illustrates a specific example of an attribute relationship table of FIG. 1.

In FIG. 3, an attribute Pr related with an attribute P is registered in the attribute relationship table 32. For example, a medicine and a symptom are registered as the attribute Pr related with the attribute P of the disease. In addition, a disease, a symptom, and an effect are registered as the attribute Pr related with the attribute P of the medicine.

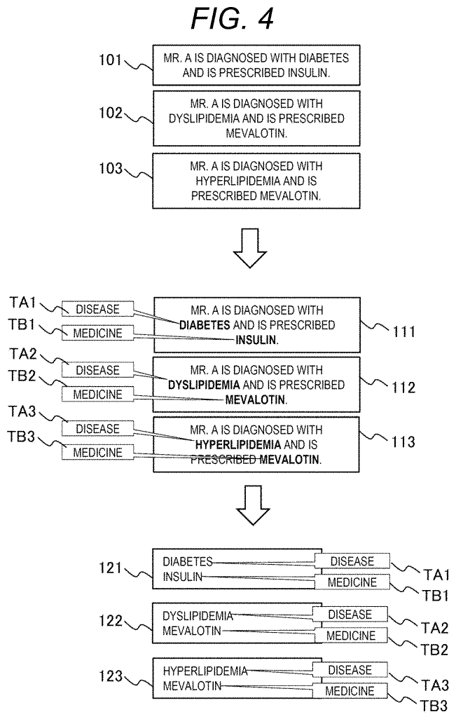

FIG. 4 illustrates a specific example of the result of attaching an attribute tag to a word having an attribute selected from the attribute relationship table of FIG. 1.

In FIG. 4, it is assumed that document data 101 to document data 103 are given to determine a synonym in the medical field. At this time, the CPU 11 refers to the word-attribute correspondence table 33 of FIG. 2 to extract words registered in the word-attribute correspondence table 33 from the document data 101 to the document data 103. Then, the CPU 11 generates attribute tagged document data 111 to attribute tagged document data 113 by attaching an attribute tag indicating an attribute registered in the word-attribute correspondence table 33 to the words extracted from the document data 101 to the document data 103.

For example, an attribute tag TA1 which indicates a disease attribute is attached to the word diabetes, and an attribute tag TB1 which indicates a medicine attribute is attached to the word insulin in the attribute tagged document data 111. An attribute tag TA2 which indicates a disease attribute is attached to the word dyslipidemia, and an attribute tag TB2 which indicates a medicine attribute is attached to the word mevalotin in the attribute tagged document data 112. An attribute tag TA3 which indicates a disease attribute is attached to the word hyperlipidemia, and an attribute tag TB3 which indicates a medicine attribute is attached to the word mevalotin in the attribute tagged document data 113.

The attribute tagged document data 111 to the attribute tagged document data 113 can be held in the form of the document-attribute tag correspondence table 27 of FIG. 1.

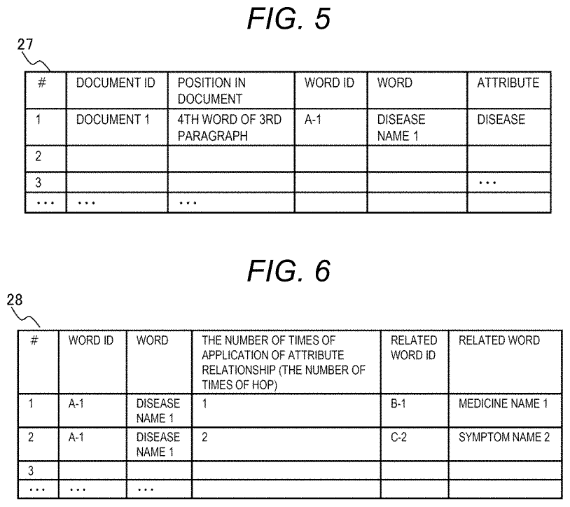

FIG. 5 illustrates a specific example of a document-attribute tag correspondence table of FIG. 1.

In FIG. 5, a document ID for identifying a document from which a word is extracted, a position of the word in the document, a word ID for identifying the word extracted from the document, and a word extracted from a document and an attribute of the word are registered in the document-attribute tag correspondence table 27.

Next, the CPU 11 generates deleted attribute tagged document data 121 to deleted attribute tagged document data 123, in which words to which the attribute tag TA1 to the attribute tag TA3 and the attribute tag TB1 to the attribute tag TB3 are not attached are deleted from the attribute tagged document data 111 to the attribute tagged document data 113 of FIG. 4.

The deleted attribute tagged document data 121 to the deleted attribute tagged document data 123 can be held in the form of the related word correspondence table 28 of FIG. 1.

FIG. 6 illustrates a specific example of a related word correspondence table of FIG. 1.

In FIG. 6, a word ID for identifying a word, a word, a related word ID for identifying a related word, a related word, and the number of times of application of attribute relationship (the number of times of hop) are registered in the related word correspondence table 28. The related word is a word having an attribute related with the attribute of the word. The number of times of application of attribute relationship is the number of times the attribute relationship registered in the attribute relationship table 32 of FIG. 3 is applied.

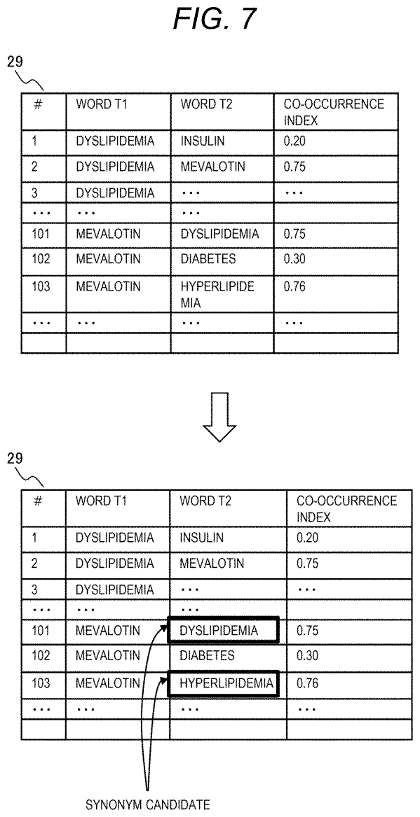

FIG. 7 illustrates a specific example of a synonym candidate extracted from a word co-occurrence index calculation result of FIG. 1.

In FIG. 7, the CPU 11 of FIG. 1 calculates the co-occurrence index between a word T1 and a word T2 having attributes related with each other by applying, for example, the word2vec to the deleted attribute tagged document data 121 to the deleted attribute tagged document data 123 of FIG. 4. For example, the co-occurrence index between the word T1 dyslipidemia and the word T2 insulin is calculated to be 0.20, and the co-occurrence index between the word T1 dyslipidemia and the word T2 mevalotin is calculated to be 0.75.

Here, by calculating the co-occurrence index between the word T1 and the word T2 for the deleted attribute tagged document data 121 to the deleted attribute tagged document data 123 of FIG. 4, the load of the calculation can be reduced compared with the method of calculating the co-occurrence index between the word T1 and the word T2 for the document data 111 to the document data 113.

Then, when there is a set of words T2 having the same attribute among words whose co-occurrence index calculated between the word T1 and the word T2 having attributes related with each other is equal to or more than a lower limit value, the CPU 11 determines the set of words T2 to be synonymous.

For example, it is assumed that the lower limit value of the co-occurrence index is 0.7. Then, the attribute of words T2 dyslipidemia and hyperlipidemia is a disease, and the attribute of the word T1 mevalotin is a medicine. By referring to the attribute relationship table 32 of FIG. 3, it is determined that the disease and the medicine are attributes related with each other. In addition, the co-occurrence index between the word T1 mevalotin and the word T2 dyslipidemia is 0.75, and the co-occurrence index between the word T1 mevalotin and the word T2 hyperlipidemia is 0.76. Therefore, the co-occurrence indexes calculated between the word T1 mevalotin and the words T2 dyslipidemia and hyperlipidemia are equal to or more than the lower limit value, and the words T2 dyslipidemia and hyperlipidemia have the same attribute of disease. Therefore, the CPU 11 determines dyslipidemia and hyperlipidemia to be synonymous, and dyslipidemia and hyperlipidemia can be considered as synonym candidates.

Next, the CPU 11 presents the word hyperlipidemia to the user 3 as a synonym candidate of the word dyslipidemda. Then, when the user 3 determines that the word hyperlipidemia is a synonym of the word dyslipidemia and performs a registration instruction, the CPU 11 registers the word hyperlipidemia in the synonym dictionary 34 as a synonym of the word dyslipidemia.

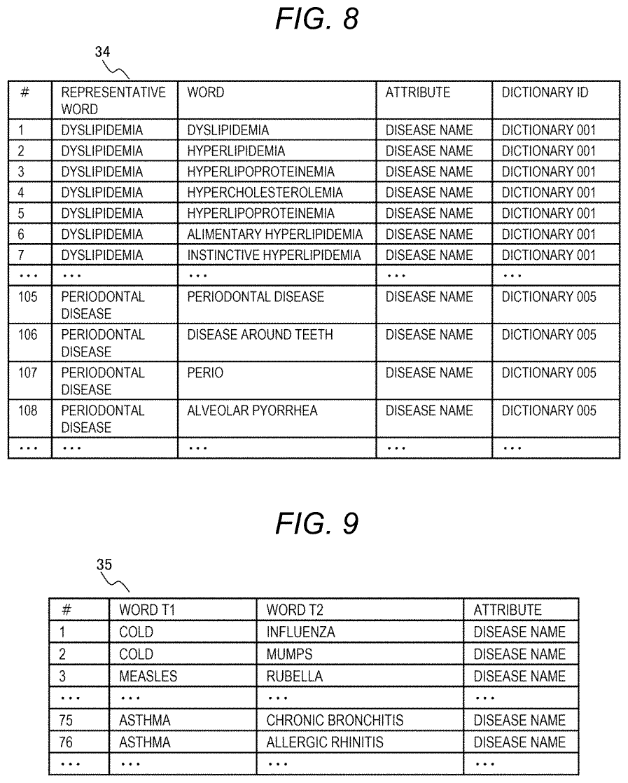

FIG. 8 illustrates a specific example of a synonym dictionary of FIG. 1.

In FIG. 8, a representative word which represents a synonym, a word synonymous with the representative word, a word attribute, and a dictionary ID for identifying a synonym dictionary are registered in the synonym dictionary 34. For example, words hyperlipidemia, hypertriglyceridemia, hypercholesterolemia, hyperlipoproteinemia, alimentary hyperlipidemia, and instinctive hyperlipidemia are registered as synonyms for the word dyslipidemia. In addition, as synonyms of the word periodontal disease, words disease around teeth, perio and alveolar pyorrhea are registered.

On the other hand, the CPU 11 determines, for example, the word cold and the word influenza to be synonymous, and presents them to the user as synonym candidates. Then, when the user 3 determines that the word cold is not a synonym of the word influenza and performs an unregistered instruction, the CPU 11 registers the word influenza in the synonym exclusion list 35 as a non-synonym of the word cold.

FIG. 9 illustrates a specific example of a synonym exclusion list of FIG. 1.

In FIG. 9, a word T1 and a word T2 which are not synonyms with each other and attributes of the word T1 and the word T2 are registered in the synonym exclusion list 35. For example, words T2 influenza and mumps are registered as non-synonyms of the word T1 cold, the word T2 rubella is registered as a non-synonym of the word T1 measles, and words T2 chronic bronchitis and allergic rhinitis are registered as non-synonyms of the word T1 asthma.

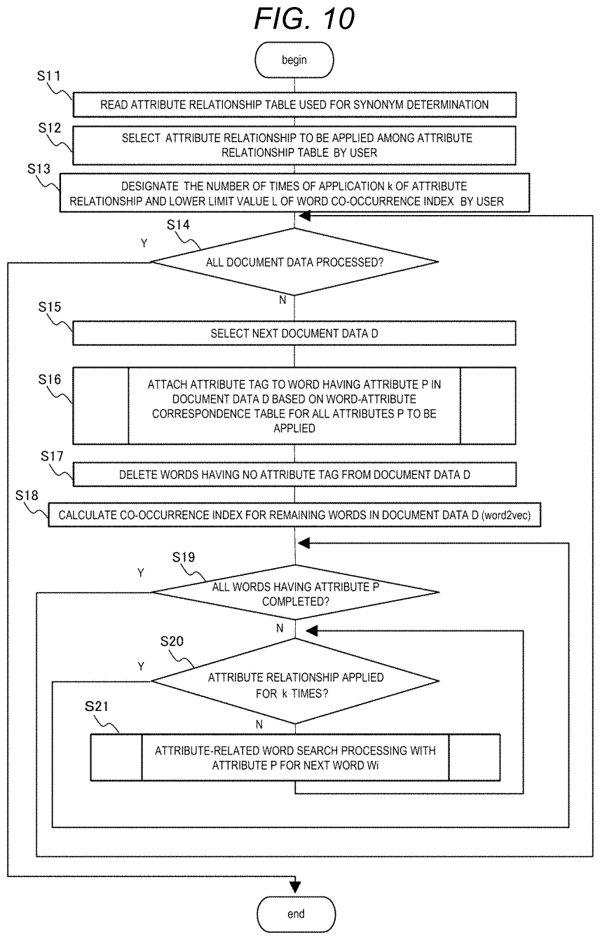

FIG. 10 is a flowchart illustrating a synonym dictionary update processing of the document search system of FIG. 1.

In FIG. 10, the CPU 11 of FIG. 1 reads the attribute relationship table 32 used for synonym determination (S11). Then, the CPU 11 displays attribute relationship registered in the attribute relationship table 32 on the terminal 4 of FIG. 1.

Next, the user 3 selects attribute relationship to be applied to a current synonym determination from the attribute relationship displayed on the terminal 4 (S12). Next, the user 3 designates the number of times of application k (k is a positive integer) of the attribute relationship and a lower limit value L of word co-occurrence index to be applied to the current synonym determination on the terminal 4 (S13).

Next, the CPU 11 determines whether all document data is processed (S14). When all document data is processed, the CPU 11 ends the synonym dictionary update processing. On the other hand, when all document data is not processed, the CPU 11 selects next document data D (S15).

Next, the CPU 11 refers to the word-attribute correspondence table 33 of FIG. 1 to attach an attribute tag to a word having an attribute P in the document data D for all the attributes P to be applied to the synonym determination (S16). At this time, the CPU 11 registers the position of the word in the document data D to which the attribute tag is attached in the document-attribute tag correspondence table 27 of FIG. 5.

Next, the CPU 11 deletes words to which no attribute tag is attached from the document data D (S17).

Next, the CPU 11 calculates a co-occurrence index for the remaining words in the document data D, in which words to which no attribute tag is attached is deleted, by using, for example, the word2vec (S18).

Next, the CPU 11 determines whether the co-occurrence index is calculated for all words having the attribute P in the document data D (S19). When the CPU 11 determines that the co-occurrence index is calculated for all the words having the attribute P in the document data D, the processing returns to S14. On the other hand, when the co-occurrence index is not calculated for all the words having the attribute P in the document data D, the CPU 11 determines whether the attribute relationship is applied for only the number of times of application k (S20). When the CPU 11 determines that the attribute relationship is applied for only the number of times of application k, the processing returns to S19. When the attribute relationship is not applied for only the number of times of application k, the CPU 11 executes attribute-related word search processing with the attribute P for the next word Wi (S21), and the processing returns to S20.

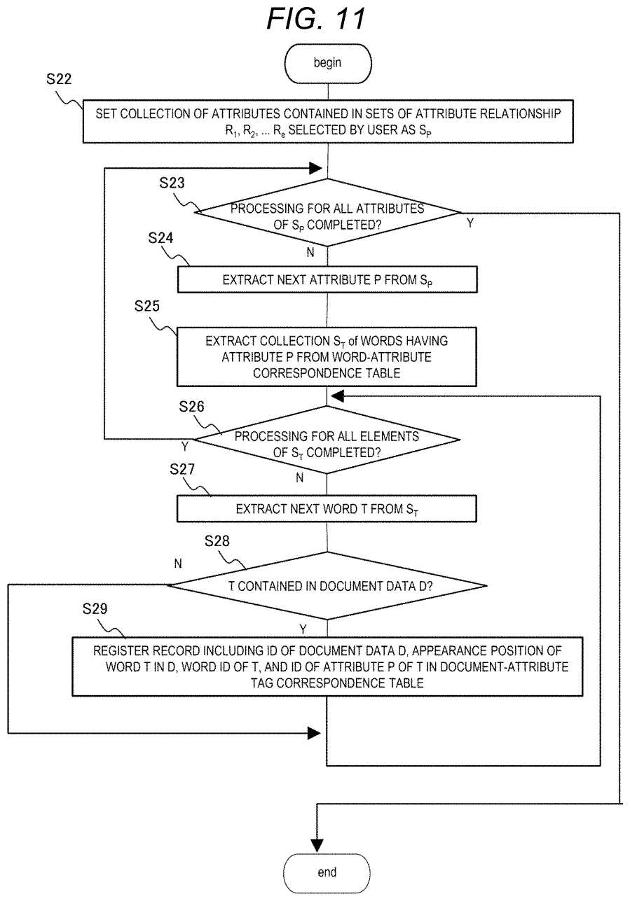

FIG. 11 is a flowchart illustrating an attribute tag attaching processing of the document search system of FIG. 1. The CPU 11 can call the attribute tag attaching processing of FIG. 11 in S16 of FIG. 10.

In FIG. 11, the CPU 11 sets a collection of attributes contained in sets of attribute relationship R1, R2, . . . Rₑ (e is a positive integer) selected by the user 3 as S_{P} (S22).

Next, the CPU 11 determines whether the attribute tag attaching processing is completed for all the attributes contained in the attribute collection S_{P} (S23). When the attribute tag attaching processing is completed for all the attributes included in the attribute collection S_{P}, the CPU 11 ends the attribute tag attaching processing. On the other hand, when the attribute tag attaching processing is not completed for all the attributes included in the attribute collection S_{P}, the CPU 11 extracts a next attribute P from the attribute collection S_{P} (S24).

Next, the CPU 11 extracts a collection S_{T} of words having the attribute P from the word-attribute correspondence table 33 of FIG. 1 (S25).

Next, the CPU 11 determines whether the attribute tag attaching processing is completed for all elements of the word collection ST (S26). When the CPU 11 determines that the attribute tag attaching processing is completed for all elements of the word collection S_{T}, the processing returns to S23. On the other hand, when the attribute tag attaching processing is not completed for all elements of the word collection S_{T}, the CPU 11 extracts a next word T from the word collection S_{T} (S27).

Next, the CPU 11 determines whether the word T is contained in the document data D selected in S15 of FIG. 10 (S28). When the CPU 11 determines that the word T is not contained in the document data D, the processing returns to S26. On the other hand, when the word T is contained in the document data D, the CPU 11 registers a record including a document ID of the document data D, an appearance position of the word T in the document data D, a word ID of the word T, and an ID of the attribute P of the word T in the document-attribute tag correspondence table 27 (S29), and the processing returns to S26.

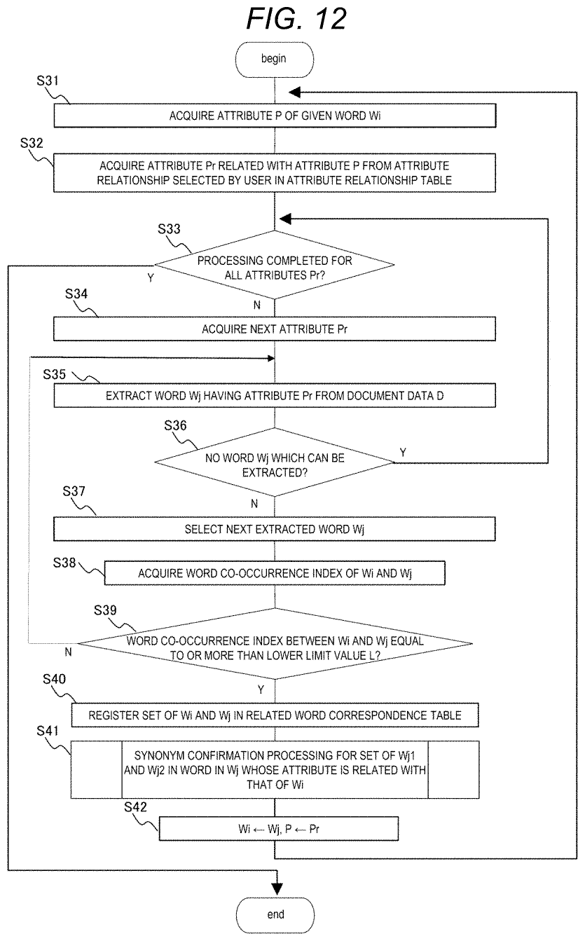

FIG. 12 is a flowchart illustrating an attribute-related word search processing of the document search system of FIG. 1. The CPU 11 can call the attribute-related word search processing of FIG. 12 in S21 of FIG. 10.

In FIG. 12, the CPU 11 acquires the attribute P of the word Wi given in S21 of FIG. 10 by referring to the word-attribute correspondence table 33 of FIG. 1 (S31).

Next, the CPU 11 acquires the attribute Pr related with the attribute P from the attribute relationship selected by the user 3 in the attribute relationship table 32 of FIG. 1 (S32).

Next, the CPU 11 determines whether the attribute-related word search processing is completed for all the attributes Pr (S33). When the attribute-related word search processing is completed for all the attributes Pr, the CPU 11 ends the attribute-related word search processing. On the other hand, when the attribute-related word search processing is not completed for all the attributes Pr, the CPU 11 acquires a next attribute Pr (S34).

Next, the CPU 11 extracts a word Wj having the attribute Pr from the document data D (S35). At this time, the CPU 11 determines whether there is a word Wj which can be extracted from the document data D (S36). When the CPU 11 determines that there is no word Wj which can be extracted from the document data D, the processing returns to S33. On the other hand, when there is a word Wj which can be extracted from the document data D, the CPU 11 selects a next extracted word Wj (S37).

Next, the CPU 11 acquires a co-occurrence index of the word Wi and the word Wj from the word co-occurrence index calculation result 29 of FIG. 1 (S38).

Next, the CPU 11 determines whether the co-occurrence index between the word Wi and the word Wj is equal to or more than the lower limit value L (S39). When the CPU 11 determines that the co-occurrence index between the word Wi and the word Wj is not equal to or more than the lower limit value L, the processing returns to S35. On the other hand, when the co-occurrence index between the word Wi and the word Wj is equal to or more than the lower limit value L, the CPU 11 registers a set of the word Wi and the word Wj in the related word correspondence table 28 of FIG. 1 (S40).

Next, the CPU 11 executes synonym confirmation processing for a set of a word Wj1 and a word Wj2 in the word Wj whose attribute is related with that of the word Wi (S41).

Next, the CPU 11 sets the Wj as Wi and sets Pr as P, and the processing returns to S31 (S42).

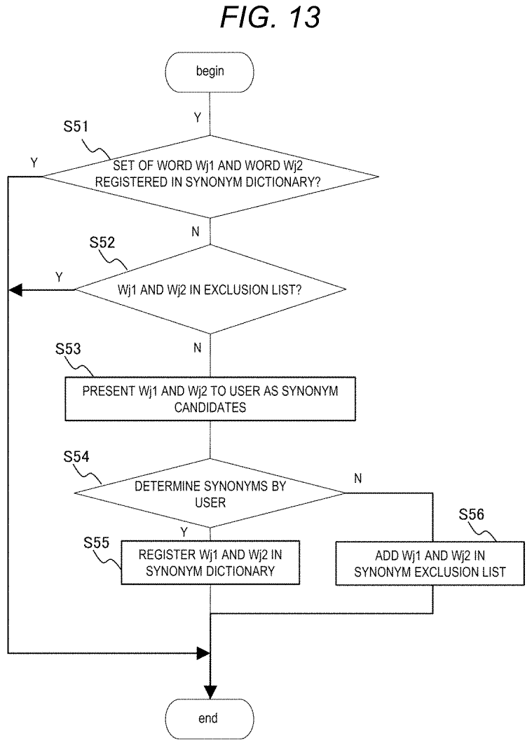

FIG. 13 is a flowchart illustrating a synonym registration processing of the document search system of FIG. 1. The CPU 11 can call the synonym registration processing of FIG. 13 in S41 of FIG. 12.

In FIG. 13, the CPU 11 determines whether the set of the word Wj1 and the word Wj2 is registered in the synonym dictionary 34 of FIG. 1 (S51). When the set of the word Wj1 and the word Wj2 is registered in the synonym dictionary 34, the CPU 11 ends the synonym registration processing.

On the other hand, when the set of the word Wj1 and the word Wj2 is not registered in the synonym dictionary 34, the CPU 11 determines whether the set of the word Wj1 and the word Wj2 is registered in the synonym exclusion list 35 of FIG. 1 (S52). When the set of the word Wj1 and the word Wj2 is registered in the synonym exclusion list 35 of FIG. 1, the synonym registration processing is ended.

On the other hand, when the set of the word Wj1 and the word Wj2 is not registered in the synonym exclusion list 35 of FIG. 1, the CPU 11 presents the set of the word Wj1 and the word Wj2 to the user 3 as synonym candidates (S53).

Next, the user 3 determines whether the set of the word Wj1 and the word Wj2 are synonyms (S54). When the user 3 determines that the set of the word Wj1 and the word Wj2 are synonyms, the CPU 11 registers the set of the word Wj1 and the word Wj2 as synonyms in the synonym dictionary 34 (S55). On the other hand, when the user 3 determines that the set of the word Wj1 and the word Wj2 are not synonyms, the CPU 11 registers the set of the word Wj1 and the word Wj2 as non-synonyms in the synonym exclusion list 35 (S56).

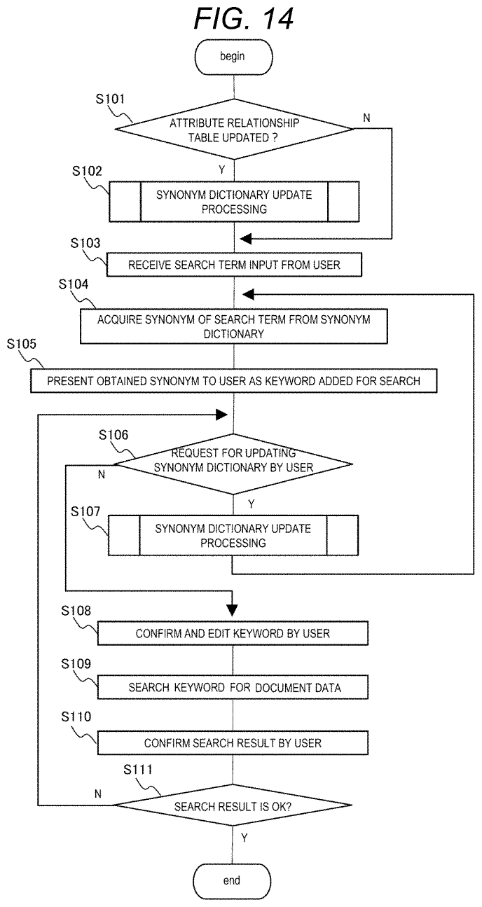

FIG. 14 is a flowchart illustrating a document search processing of the document search system of FIG. 1.

In FIG. 14, the CPU 11 of FIG. 1 determines whether the attribute relationship table 32 is updated (S101). When the CPU 11 determines that the attribute relationship table 32 is not updated, the processing proceeds to S103. On the other hand, when the attribute relationship table 32 is updated, the CPU 11 executes the synonym dictionary update processing of FIG. 10 (S102).

Next, when receiving a search term input from the user 3 (S103), the CPU 11 acquires a synonym of the search term from the synonym dictionary 34 (S104).

Next, the CPU 11 presents the synonym obtained from the synonym dictionary 34 to the user 3 as a keyword added for the search (S105).

Next, the CPU 11 determines whether there is a request for updating the synonym dictionary 34 by the user 3 (S106). When there is a request for updating the synonym dictionary 34 by the user 3, the CPU 11 executes the synonym dictionary update processing of FIG. 10 (S107), and the processing returns to S104. On the other hand, when there is no request for updating the synonym dictionary 34 by the user 3, the CPU 11 waits until the user 3 confirms and edits the keyword (S108).

Next, when the user 3 confirms and edits the keyword, the CPU 11 searches the document data 31 for the keyword (S109), and presents the search result of the document data 31 to the user 3.

Next, the user 3 confirms the search result of the document data 31 (S110). Then, the user 3 determines whether the search result is OK (S111). When the user 3 issues an instruction that the search result is OK, the CPU 11 ends the document search processing. On the other hand, when the user 3 issues an instruction that the search result is not OK, the processing returns to S106 by the CPU 11.

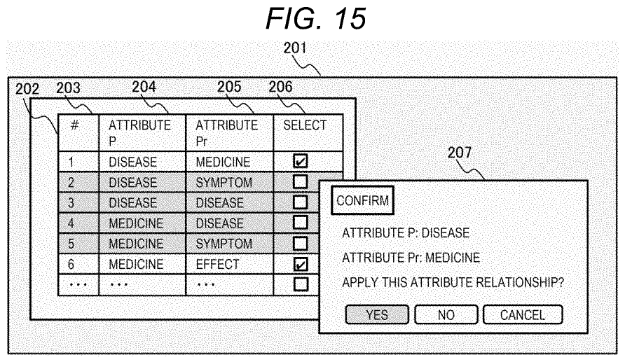

FIG. 15 illustrates a specific example of an attribute relationship selection screen displayed on a terminal of FIG. 1. An attribute relationship selection screen 202 is displayed on a display screen 201 of the terminal 4 when the user 3 performs the operation of S12 of FIG. 10.

In FIG. 15, an identification number 203 for identifying an attribute relationship, a display column 204 and a display column 205 of the set of the attribute P and the attribute Pr related with the attribute P, and a check box 206 for selecting an attribute relationship are displayed in the attribute relationship selection screen 202.

For example, when the user 3 selects the medicine as the attribute Pr related with the attribute P of disease, the check box 206 corresponding to 1 of the identification number 203 is checked. When the check box 206 is checked, the CPU 11 displays a selection confirmation screen 207 on the display screen 201. When the user 3 selects “YES” on the selection confirmation screen 207 and presses a confirm button, the CPU 11 applies this attribute relationship in the synonym dictionary update processing of FIG. 10.

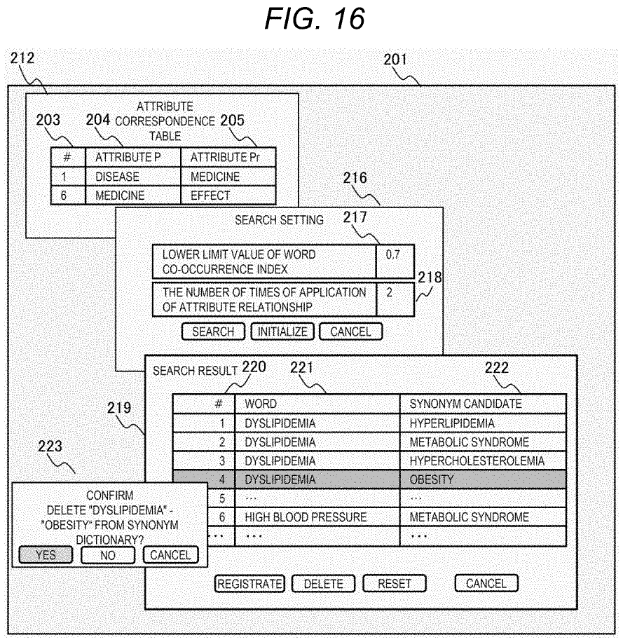

FIG. 16 illustrates a specific example of a search condition setting screen and a search result screen displayed on the terminal of FIG. 1. A search condition setting screen 216 is displayed on the display screen 201 of the terminal 4 when the user 3 performs the operation of S13 of FIG. 10. A search result screen 219 is displayed on the display screen 201 of the terminal 4 when the CPU 11 executes the processing of S53 of FIG. 13.

In FIG. 16, when the user 3 selects the attribute relationship corresponding to 1 and 6 of identification numbers 203 on the attribute relationship selection screen 202 of FIG. 15, an attribute relationship selection result screen 212 showing the attribute relationship corresponding to 1 and 6 of the identification numbers 203 is displayed on the display screen 201.

In addition, the search condition setting screen 216 is displayed on the display screen 201 together with the attribute relationship selection result screen 212. An input column 217 of a lower limit value of word co-occurrence index and an input column 218 of the number of times of application of attribute relationship are displayed on the search condition setting screen 216. Then, for example, the user 3 can set the lower limit value of word co-occurrence index to 0.7 and the number of times of application of attribute relationship to 2 on the search condition setting screen 216.

When the lower limit value of word co-occurrence index and the number of times of application of attribute relationship are set, the CPU 11 searches for a synonym candidate for a certain word in the synonym dictionary update processing of FIG. 10. Then, the CPU 11 presents the synonym candidate for the certain word to the user 3 by displaying the synonym search result screen 219 on the display screen 201 in the processing of S53 of FIG. 13. An identification number 220 for identifying a set of a certain word and a synonym candidate, and a display column 221 and a display column 222 of a set of a word and a synonym candidate are displayed in the synonym search result screen 219.

For example, when the user 3 selects a set of dyslipidemia and obesity on the synonym search result screen 219 and presses a delete button, the CPU 11 displays a deletion confirmation screen 223 on the display screen 201. When the user 3 selects “YES” on the deletion confirmation screen 223, the CPU 11 deletes the set of “dyslipidemia” and “obesity” from the synonym dictionary 34.

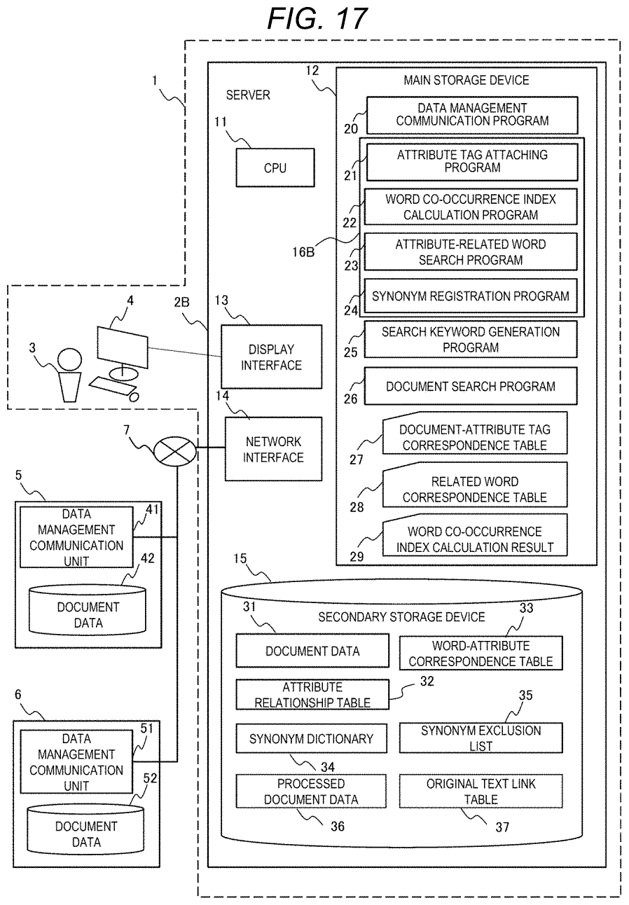

FIG. 17 is a block diagram illustrating a configuration of a document search system according to a second embodiment.

In FIG. 17, the document search system includes a server 2B instead of the server 2A of FIG. 1. The server 2B has the same configuration as that of the server 2A.

However, the main storage device 12 of the server 2B holds a synonym dictionary update processing program 16B instead of the synonym dictionary update processing program 16A of FIG. 1. The synonym dictionary update processing program 16B realizes the same processing as the synonym dictionary update processing program 16A. However, when the synonym dictionary update processing program 16B deletes words to which no attribute tag is attached from the document data D in S17 of FIG. 10, an access destination of an original text before deleting the word is held. In addition, the secondary storage device 15 of the server 2B holds a processed document data 36 and an original text link table 37 in addition to storage contents of the secondary storage device 15 of the server 2A.

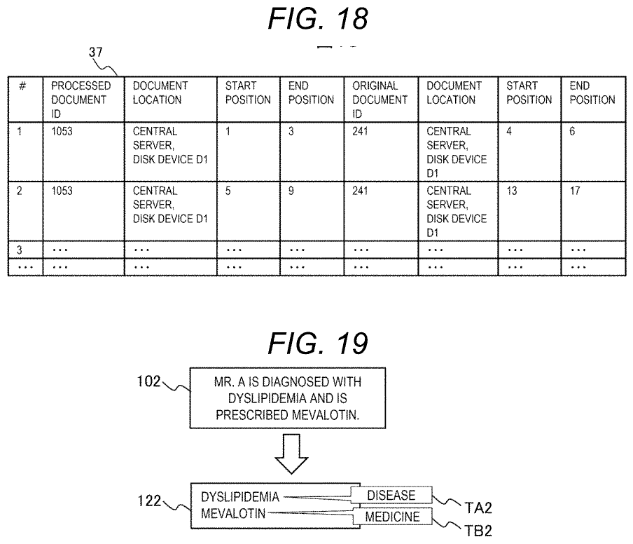

FIG. 18 illustrates a specific example of the original text link table of FIG. 17.

In FIG. 18, a processing document ID for identifying a processed document, a document location where the processed document is stored, a storage start position of the processed document, a storage end position of the processed document, an original document ID for identifying an original document, a document location where the original document is stored, a storage start position of the original document, and a storage end position of the original document are registered in the original text link table 37. The document location can be designated by a device name for storing data and may be, for example, a central server or a disk device D1.

FIG. 19 illustrates a specific example of the original document and the processed document used in the document search system of FIG. 17.

In FIG. 19, it is assumed that the CPU 11 generates, for example, the deleted attribute tagged document data 122 from the document data 102. At this time, for example, the CPU 11 attaches the original document ID=241 to the document data 102, and registers information that the document data 102 is stored from the start position=4 to the end position=6 of the central server and the disk device D1 in the original text link table 37. For example, the CPU 11 attaches the processed document ID=1053 to the deleted attribute tagged document data 122, and registers information that the deleted attribute tagged document data 122 is stored from the start position=1 to the end position=3 of the central server and the disk device D1 in the original text link table 37.

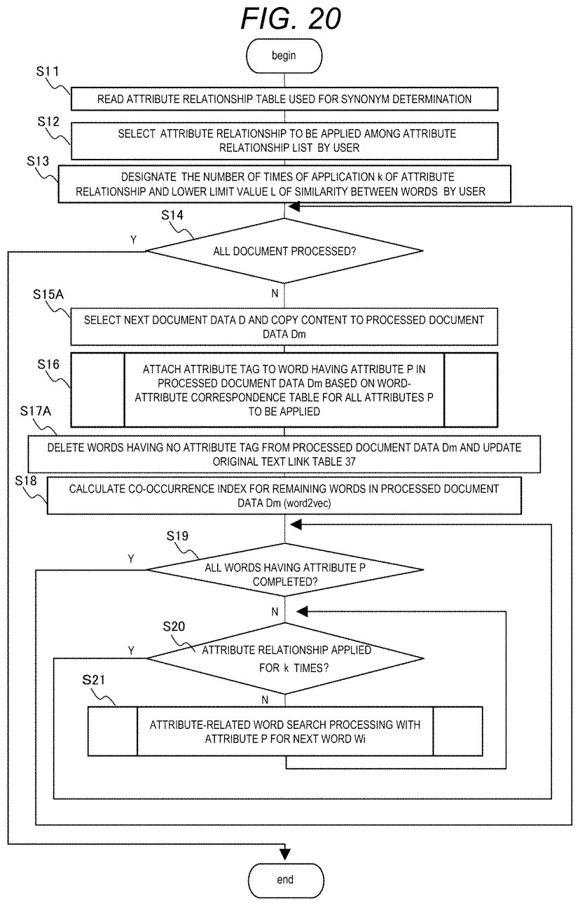

FIG. 20 is a flowchart illustrating a synonym dictionary update processing of the document search system of FIG. 17.

The synonym dictionary update processing in FIG. 20 includes S15A and S17A instead of S15 and S17 of the synonym dictionary update processing in FIG. 10.

In S15A, processing of copying the content of the document data D to a processed document data Dm is added to the processing of selecting the next document data D in S15. In S17A, processing of updating the original text link table 37 in FIG. 17 is added to the processing of deleting words to which no attribute tag is attached from the document data D in S17.

By holding the original text link table 37, the server 2B can access the document data D before the words to which no attribute tag is attached are deleted even when the words to which no attribute tag is attached are deleted from the document data D.

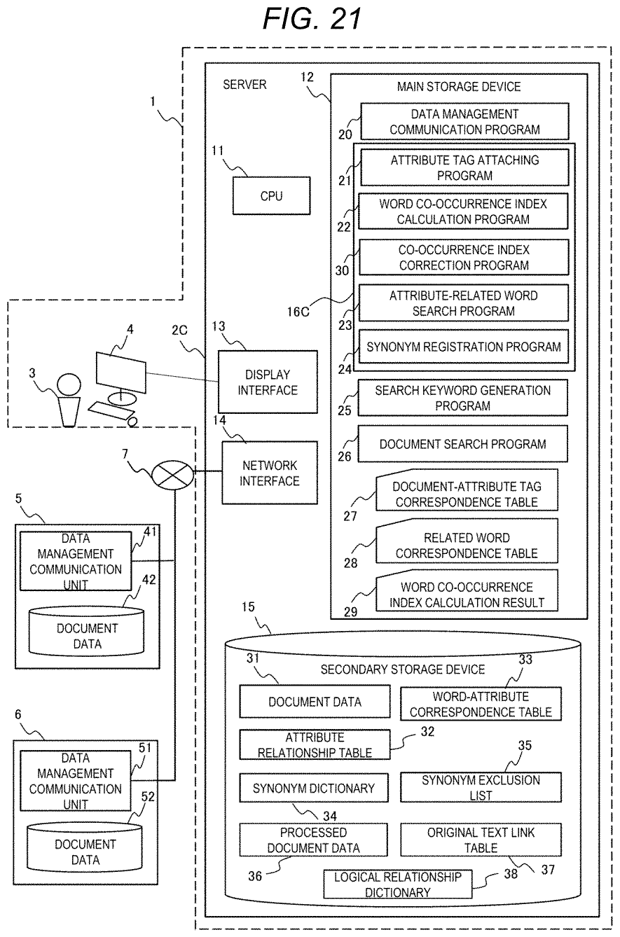

FIG. 21 is a block diagram illustrating a configuration of a document search system according to a third embodiment.

In FIG. 21, the document search system includes a server 2C instead of the server 2A of FIG. 1. The server 2C has the same configuration as that of the server 2A.

However, the main storage device 12 of the server 2C holds a synonym dictionary update processing program 16C instead of the synonym dictionary update processing program 16A of FIG. 1. The synonym dictionary update processing program 16C realizes the same processing as the synonym dictionary update processing program 16A. However, when the synonym dictionary update processing program 16C determines whether words extracted from a document are synonyms, attributes of the words, a co-occurrence index between the words, and logical relationship between the words are referred to. The logical relationship between the words is, for example, a dependency (part-of) between the words or a synonymous relationship (is-a) between the words.

In this case, a co-occurrence index correction program 30 can be added to the synonym dictionary update processing program 16C. The co-occurrence index correction program 30 corrects the co-occurrence index between the words based on the logical relationship between the words. The secondary storage device 15 of the server 2C holds a logical relationship dictionary 38 in addition to storage contents of the secondary storage device 15 of the server 2B. The logical relationship dictionary 38 registers a set of words having logical relationship.

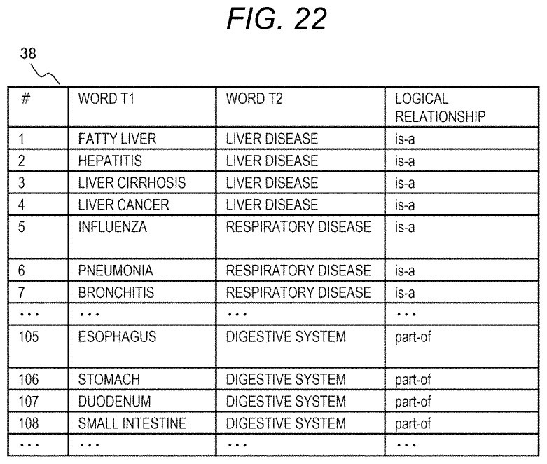

FIG. 22 illustrates a specific example of the logical relationship dictionary of FIG. 21.

In FIG. 22, a set of a word T1 and a word T2 having logical relationship is registered in the logical relationship dictionary 38. For example, logical relationship is-a is registered in the logical relationship dictionary 38 for the word T1 fatty liver and the word T2 liver disease, and logical relationship part-of is registered for the word T1 esophagus and the word T2 digestive system.

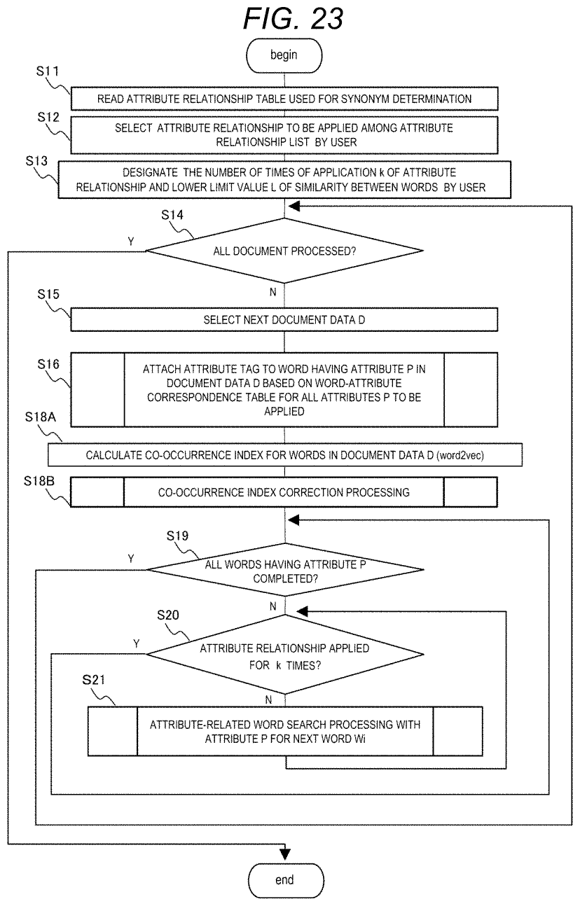

FIG. 23 is a flowchart illustrating a synonym dictionary update processing of the document search system of FIG. 21.

The synonym dictionary update processing of FIG. 23 includes S18A and S18B instead of S18 of the synonym dictionary update processing of FIG. 10, and S17 of the synonym dictionary update processing of FIG. 10 is removed.

In S18 of the synonym dictionary update processing of FIG. 10, a co-occurrence index is calculated for remaining words in the document data D in which words to which no attribute tag is attached are deleted. However, in S18A of the synonym dictionary update processing of FIG. 23, the co-occurrence index is calculated for words in the document data D before words to which no attribute tag is attached are deleted.

By calculating the co-occurrence index for the words in the document data D before the words to which no attribute tag is attached are deleted, the co-occurrence index between the words to which no attribute tag is attached can be reflected in the calculation of the co-occurrence index between words to which the attribute tag is attached, and the calculation accuracy of the co-occurrence index of the words to which the attribute tag is attached can be improved.

Next, the CPU 11 refers to the logical relationship dictionary 38 to correct the co-occurrence index calculated for the words in the document data D (S18B).

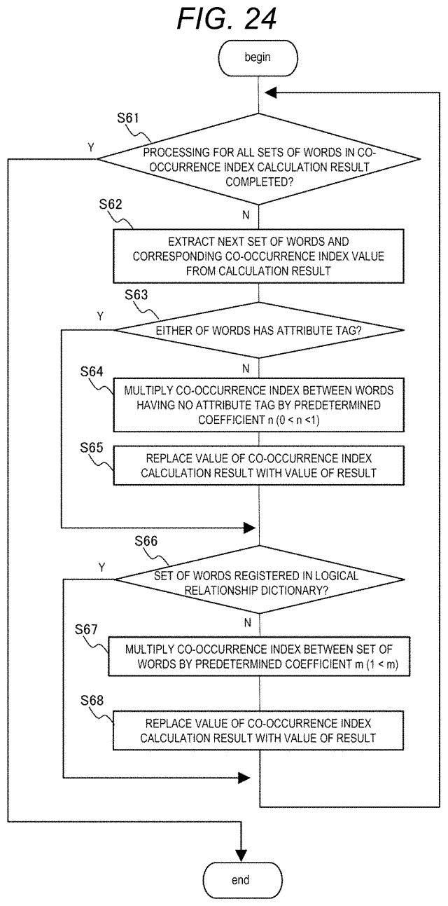

FIG. 24 is a flowchart illustrating a co-occurrence index correction processing of the document search system of FIG. 21.

In FIG. 24, the CPU 11 of FIG. 21 determines whether the co-occurrence index correction processing is executed for all sets of words for which the co-occurrence index is calculated in S18A of FIG. 23 (S61). When the co-occurrence index correction processing is executed for all sets of words, the CPU 11 ends the co-occurrence index correction processing. On the other hand, when the co-occurrence index correction processing is not executed for all sets of words, the CPU 11 extracts a next set of words for which the co-occurrence index is calculated and the co-occurrence index of this set of words from the word co-occurrence index calculation result 29 (S62).

Next, the CPU 11 determines whether either of the words extracted in S62 has an attribute tag (S63). When the CPU 11 determines both of the words have an attribute tag, the processing proceeds to S66. On the other hand, when neither of the words has an attribute tag, the CPU 11 multiplies the co-occurrence index between the words having no attribute tag by a predetermined coefficient n (0<n<1) (S64), and replaces the value of the word co-occurrence index calculation result 29 with the value of the calculation result of S64 (S65).

Next, the CPU 11 determines whether the set of words extracted in S62 is registered in the logical relationship dictionary 38 (S66). When the CPU 11 determines that the set of words is registered in the logical relationship dictionary 38, the processing returns to S61. On the other hand, when the set of words is not registered in the logical relationship dictionary 38, the CPU 11 multiplies the co-occurrence index between the set of words by a predetermined coefficient m (1<m) (S67), and replaces the value of the word co-occurrence index calculation result 29 with the value of the calculation result of S67 (S68).

In the third embodiment described above, the method of calculating the co-occurrence index for words in the document data D before words to which no attribute tag is attached are deleted is described in S18A of the synonym dictionary update processing of FIG. 23. However, as in S18 of the synonym dictionary update processing of FIGS. 10 and 20, the co-occurrence index may be calculated for the remaining words in the document data D in which the words to which no attribute tag is attached are deleted. In this case, the processing from S63 to S65 can be omitted in the co-occurrence index correction processing of FIG. 24.

In the first embodiment and the second embodiment described above, the method of calculating the co-occurrence index for the remaining words in the document data D in which words to which no attribute tag is attached are deleted is described in S18 of the synonym dictionary update processing of FIGS. 10 and 20. However, as in S18A of the synonym dictionary update processing of FIG. 23, the co-occurrence index may be calculated for words in the document data D before the words to which no attribute tag is attached are deleted.

The third embodiment described above describes the method of determining words to be synonymous based on attributes of the words, the co-occurrence index between the words, and the logical relationship between the words. However, the words may be determined to be synonymous based on the co-occurrence index between the words and the logical relationship between the words. At this time, when the co-occurrence index between the words having the logical relationship is equal to or more than the lower limit value, these words can be determined to be synonymous.

In the above description, a document search method in the medical field is described as an example. However, the invention may be applied to a document search method other than in the medical field such as in equipment maintenance.

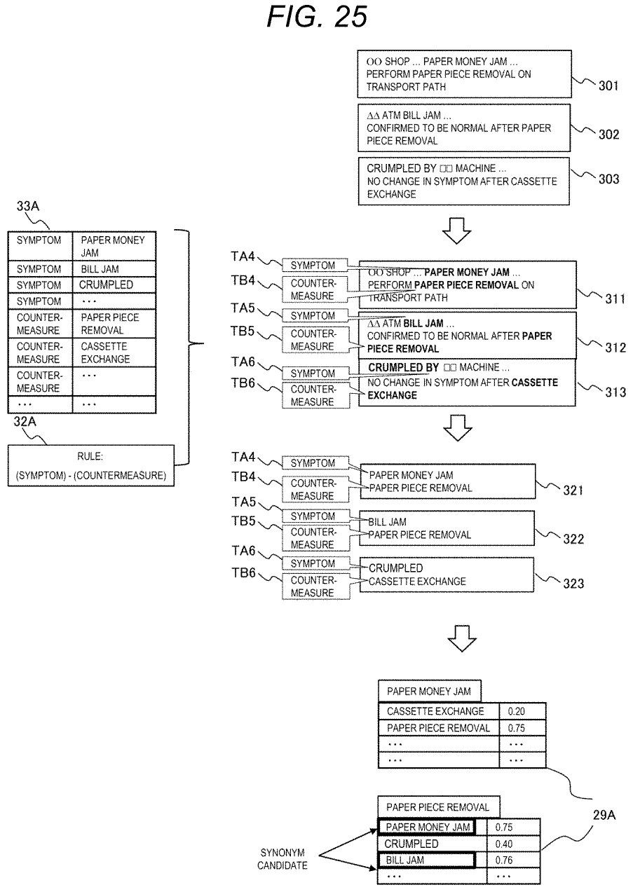

FIG. 25 illustrates an example of extraction of a synonym candidate of a document search system according to a fourth embodiment.

In FIG. 25, a symptom attribute is registered corresponding to a word which indicates a symptom name such as paper money jam, bill jam, and crumpled in a word-attribute correspondence table 33A. A countermeasure attribute is registered corresponding to a word which indicates a countermeasure name such as paper piece removal and cassette exchange in the word-attribute correspondence table 33A. The symptom and countermeasure are registered as attributes related with each other in the attribute relationship table 32A.

It is assumed that document data 301 to document data 303 are given to determine synonyms in the field of equipment maintenance. At this time, the CPU 11 refers to the word-attribute correspondence table 33A to extract words registered in the word-attribute correspondence table 33A from the document data 301 to the document data 303. Then, the CPU 11 generates attribute tagged document data 311 to attribute tagged document data 313 by attaching an attribute tag which indicates an attribute registered in the word-attribute correspondence table 33A to the words extracted from the document data 301 to the document data 303.

For example, an attribute tag TA4 which indicates a symptom attribute is attached to the words paper money jam, and an attribute tag TB4 which indicates the countermeasure attribute is attached to the words paper piece removal in the attribute tagged document data 311. An attribute tag TA5 which indicates a symptom attribute is attached to the words bill jam, and an attribute tag TB5 which indicates a countermeasure attribute is attached to the words paper piece removal in the attribute tagged document data 312. An attribute tag TA6 which indicates a symptom attribute is attached to the word crumpled, and an attribute tag TB6 which indicates a countermeasure attribute is attached to the words cassette exchange in the attribute tagged document data 313.

Next, the CPU 11 generates deleted attribute tagged document data 321 to deleted attribute tagged document data 323 in which words to which the attribute tag TA4 to the attribute tag TA6 and the attribute tag TB4 to the attribute tag TB6 are not attached are deleted from the attribute tagged document data 311 to the attribute tagged document data 313.

Next, the CPU 11 calculates the co-occurrence index between words having attributes related with each other by applying, for example, the word2vec to the deleted attribute tagged document data 321 to the deleted attribute tagged document data 323, and stores the result in a word co-occurrence index calculation result 29A. For example, the co-occurrence index between the words paper money jam and the words cassette exchange is calculated to be 0.20, and the co-occurrence index between the words paper money jam and the words paper piece removal is calculated to be 0.75.

Then, when there is a set of words having the same attribute among words whose co-occurrence index calculated between words having attributes related with each other is equal to or more than a lower limit value, the CPU 11 determines the set of words to be synonymous.

For example, it is assumed that the lower limit value of the co-occurrence index is 0.7. Then, the attribute of words paper money jam and bill jam is a symptom, and the attribute of words paper piece removal is a countermeasure. By referring to the attribute relationship table 32A, it is determined that the symptom and the countermeasure are attributes related with each other. In addition, the co-occurrence index between the words paper piece removal and the words paper money jam is 0.75, and the co-occurrence index between the words paper piece removal and the words bill jam is 0.76. Therefore, the co-occurrence index calculated between the words paper piece removal and the words of paper money jam as well as the words bill jam is equal to or more than the lower limit value, and the words paper money jam and the words bill jam have the same attribute of symptom. Therefore, the CPU 11 determines paper money jam and bill jam to be synonymous and paper money jam and bill jam can be considered as synonym candidates.

What is claimed is:

1. A synonym determination device, wherein the synonym determination device determines, based on a co-occurrence index between a word having a first attribute and a word having a second attribute related with the first attribute, words having the second attribute in common to be synonymous.

2. The synonym determination device according to claim 1, wherein
a first co-occurrence index between a first word having the first attribute and a second word having the second attribute is calculated;
a second co-occurrence index between the first word having the first attribute and a third word having the second attribute is calculated; and
the second word and the third word are determined to be synonymous based on the first co-occurrence index and the second co-occurrence index.

3. The synonym determination device according to claim 1, comprising:
an attribute relationship table in which the second attribute related with the first attribute is registered; and
a word-attribute correspondence table in which correspondence relationship between a word and an attribute is registered.

4. The synonym determination device according to claim 2, wherein
the third word is presented as a synonym candidate of the second word when the first co-occurrence index and the second co-occurrence index are equal to or more than a lower limit value; and
the third word is registered as a synonym of the second word based on a registration instruction of the synonym candidate when the third word is presented as the synonym candidate of the second word.

5. The synonym determination device according to claim 1, wherein
to a word extracted from document data, an attribute tag which indicates an attribute of the word is attached; and
a co-occurrence index between words attached with attribute tags which indicate attributes related with each other is calculated.

6. The synonym determination device according to claim 5, wherein
the co-occurrence index is calculated for remaining words in the document data in which words to which no attribute tag is attached are deleted.

7. The synonym determination device according to claim 5, wherein
the co-occurrence index is calculated for words in the document data in which words to which no attribute tag is attached are not deleted.

8. The synonym determination device according to claim 5, wherein
the co-occurrence index between words is corrected based on logical relationship between words extracted from the document data.

9. A synonym determination device, wherein
a co-occurrence index between a first word and a second word having logical relationship is calculated; and
the first word and the second word are determined to be synonymous based on the co-occurrence index.

10. The synonym determination device according to claim 9, comprising:
a logical relationship dictionary in which the first word and the second word having the logical relationship are registered.

11. A synonym determination method in which a CPU is included, wherein
when a first attribute of a first word, and a second attribute of a second word and a third word related with the first attribute are given, the CPU determines, based on a first co-occurrence index between the first word and the second word and a second co-occurrence index between the first word and the third word, the second word and the third word to be synonymous.

12. The synonym determination method according to claim 11, wherein
when the third word is presented as a synonym candidate of the second word, the CPU registers the third word as a synonym of the second word based on a registration instruction of the synonym candidate.
